(19) United States
(12) Patent Application Publication
(10) Pub. No.: US 2014/0315192 A1
(43) Pub. Date: Oct. 23, 2014

(54) Characterizing Prostate Cancer

(71) Applicants: HAIYING WANG, BRIDGEWATER, NJ (US); SHOBHA VARDE, JACKSONVILLE, FL (US); DONDAPATI CHOWDARY, PRINCETON JUNCTION, NJ (US); JYOTI MEHROTRA, BRIDGEWATER, NJ (US); TATIANA VENER, STERLING, NJ (US); ABHIJIT MAZUMDER, BASKING RIDGE, NJ (US)

(72) Inventors: HAIYING WANG, BRIDGEWATER, NJ (US); SHOBHA VARDE, JACKSONVILLE, FL (US); DONDAPATI CHOWDARY, PRINCETON JUNCTION, NJ (US); JYOTI MEHROTRA, BRIDGEWATER, NJ (US); TATIANA VENER, STERLING, NJ (US); ABHIJIT MAZUMDER, BASKING RIDGE, NJ (US)

(73) Assignee: Veridex, LLC, Raritan, NJ (US)

(21) Appl. No.: 13/787,422

(22) Filed: Mar. 6, 2013

(51) Int. Cl. C12Q 1/68

(52) U.S. Cl. CPC C12Q 1/6886
USPC 435/6.11

(57) ABSTRACT

Methods and kits for predicting the course or aggressiveness of prostate cancer include detecting the methylation status of various genes.

### CROSS REFERENCE TO RELATED APPLICATION

This application claims the benefit of U.S. Provisional Patent Application Ser. No. 60/855,640 filed Oct. 31, 2006.

### BACKGROUND OF THE INVENTION

This invention relates to the interrogation of methylated genes in concert with other diagnostic methods and kits for use with these methods.

In higher order eukaryotes DNA is methylated only at cytosines located 5′ to guanosine in the CpG dinucleotide. This modification has important regulatory effects on gene expression, especially when it involves CpG rich areas (CpG islands) located in gene promoter regions. Abberant methylation of normally unmethylated CpG islands is a frequent event in immortalized and transformed cells and has been associated with transcriptional inactivation of certain tumor suppressor genes or genes otherwise associated with the amelioration of certain human cancers.

Glutathione S-transferases (GSTs) are exemplary proteins in which the methylation status of the genes that express them can have important prognostic and diagnostic value for prostate cancer. The proteins catalyze intracellular detoxification reactions, including the inactivation of electrophilic carcinogens, by conjugating chemically-reactive electrophiles to glutathione (C. B. Pickett, et al., Annu. Rev. Blocbern., 58:743, 1989; B. Coles, et al., CRC Crit. Rev. Biochem. Mol. Biol., 25:47, 1990; T. H. Rushmore, et al., J. Biol. Chem. 268:11475, 1993). Human GSTs, encoded by several different genes at different loci, have been classified into four families referred to as alpha, mu, pi, and theta (B. Mannervik, et al., Biochem. J., 282:305, 1992). Decreased GSTP1 expression resulting from epigenetic changes is often related to prostate and hepatic cancers.

A commonly used system for determining the prognosis of a patient with prostate cancer is the Gleason scoring system. The Gleason scoring system is based on microscopic tumor patterns assessed by a pathologist while interpreting a biopsy specimen from a patient's prostate. Nomograms have also been developed by Kattan and others in which prognosis includes the Gleason score and a number of other factors.

Gleason scores are assessed when prostate cancer is present in a prostate biopsy. The Gleason score is based upon the degree of loss of the normal glandular tissue architecture (i.e. shape, size and differentiation of the glands) as originally described and developed by Dr. Donald Gleason. See, Gleason D F, Mellinger G T, and the Veterans Administration Cooperative Urological Research Group: Prediction of prognosis for prostatic adenocarcinoma by combined histologic grading and clinical staging. J Urol 111:58-64, 1974. The classic Gleason scoring diagram shows five basic tissue patterns that are referred to as tumor “grades”. The subjective microscopic determination of this loss of normal glandular structure caused by the cancer is abstractly represented by a grade, a number ranging from 1 to 5, with 5 being the worst grade possible. The Gleason score (GS) and the Gleason sum are one and the same. However, the “Gleason grade” and the “Gleason score” (also referred to as the “Gleason sum”) are different. The Gleason score is a sum of the primary grade (representing the majority of tumor) and a secondary grade (assigned to the minority of the tumor), and is a number ranging from 2 to 10. Under current practice, it is widely held that the higher the Gleason score, the more aggressive the tumor is likely to be and the worse the patient's prognosis. While useful, the correlation between Gleason score and cancer prognosis is not straight-forward. For one thing, samples with a Gleason score of 7 or greater represent a heterogeneous group of cancers and this heterogeneity can detract from predictability. It is important to sub-stratify cancers exhibiting Gleason scores of 7 or more because the nature of the therapy provided to a patient depends upon it.

With respect to diagnostics and prognostics that do not involve biopsy samples, it is well known that Prostate Specific Antigen (“PSA”) is the standard “marker” for prostate cancer. The use of the marker is helpful but not determinative in diagnostic applications and the marker is of minimal use as a prognostic. New techniques for improving the use of known markers such as PSA would also be beneficial.

The present invention fulfills these needs.

### SUMMARY OF THE INVENTION

In one aspect of the invention, a method for characterizing prostate cancer in a patient comprises determining the Gleason score of the patient and detecting epigenetic changes such as gene methylation in the patient if his Gleason score is seven or greater. The cancer is characterized as aggressive if the degree or amount of epigenetic change exceeds a predetermined value and indolent if it does not. The patient is treated consistent with the manner in which those with aggressive or indolent prostate cancers are treated.

In one aspect of the invention, methylation of one or more genes from the following group is detected: GSTP1, APC, RASSF1A, 15-LO-1, and CDH1. Preferably, the methylation status of the GSTP1 promoter is detected in blood, a blood component, urine, urethral washings, ejaculate, or tissue sample. Most preferably, the sample is a tissue sample.

In another aspect of the invention, a Gleason score is obtained for a prostate cancer patient. If the patient is assessed as having a Gleason score of 7 or higher, another biological sample is taken from the patient or the sample from which the Gleason score was adduced is further assayed. A nucleic acid sample suspected of having methylated target sequences is obtained from one or both biological samples, the sample is treated with a reagent that can prime a portion of a nucleic acid target, the nucleic acid target is primed, and the degree of methylation of the amplified target from the sample is compared with that of a known normal sample or a predetermined value obtained from known normal samples. In yet another aspect of the invention, a sequence that is not likely to be methylated is also amplified and detected for comparison with the amplified methylated sequence.

In another aspect of the invention, methylation status is determined via quantitative real time PCR.

In yet another aspect of the invention, a method for characterizing prostate condition includes the step of first testing the patient with a screening assay such as a standard PSA assay. Those patients with concentrations of the markers that are not indicative of a condition that is likely to be cancerous but which is above a normal level are tested for methylation of a prostate cancer marker such as GSTP1, APC, RASSF1A, 15-LO-1, or CDH1. Those patients showing a methylation level beyond a predetermined level are biopsied. In a preferred aspect of this method, the methylation assay is conducted on patients having a PSA level greater than or equal to 2.5 ng/ml. Alternatively, methylation assays are conducted on those with PSA levels of 2-4.

In yet another aspect, the invention is a kit useful for the detection of a methylated nucleic acid. The kit includes one or more containers; a first container containing a reagent that modifies unmethylated cytosine and a second container containing a reagent that primes amplification of CpG-containing nucleic acid, wherein the reagent distinguishes between modified methylated and nonmethylated nucleic acid. The kit contains instructions to conduct the assay on patients with prostate samples assessed as having a Gleason score of 7 or higher. In another embodiment the instructions provide that the assay is run on patients with samples assessed as having a Gleason score greater than 7.

### DETAILED DESCRIPTION OF THE INVENTION

Gleason scores are determined on prostate tissue samples obtained from resection or biopsy. Two samples of abnormal tissue patterns are usually analyzed and their individual score is added together. Methods for sampling and assigning Gleason scores are now well known and widely practiced.

In some methods of the invention, a Gleason score is determined for a prostate cancer patient, a patient being treated for prostate cancer, or a person suspected of having prostate cancer. If the Gleason score is 7 or higher, the patient is tested to determine the methylation status of a nucleic acid that corresponds to a gene whose methylation status correlates with prostate cancer aggressiveness or progression. In the kits of the invention, instructions are provided so that methylation status of a patient is determined for patients for whom a Gleason score of 7 or higher is adduced. In other kits of the invention, instructions are provided so that methylation status of a patient is determined for patients for whom a Gleason score greater than is adduced.

A nucleic acid corresponds to a gene whose methylation status correlates with prostate cancer when methylation status of such a gene provides information about prostate cancer and the sequence is a coding portion of the gene or its complement, a representative portion of the gene or its complement, a promoter or regulatory sequence for the gene or its complement, a sequence that indicates the presence of the gene or its complement, or the full length sequence of the gene or its complement. Such nucleic acids are referred to as Markers in this specification. Markers correspond to the following genes: GSTP1 (Seq. ID. No. 59), RASSF1A (Seq. ID. No. 69), APC (Promoter=Seq. ID. No. 64, Gene=Seq. ID. No. 65), 15-LO-1 (Seq. ID. No. 56), and CDH1 (Seq. ID. No.57). Other sequences of interest include constitutive genes useful as assay controls such as beta-Actin (Seq. ID. No.60 and 61) and PTGS2 (Promoter=Seq. ID. No.66, Gene=Seq. ID. No. 67).

Assays for detecting hypermethylation include such techniques as MSP and restriction endonuclease analysis. The promoter region is a particularly noteworthy target for detecting such hypermethylation analysis. Sequence analysis of the promoter region of GSTP1 shows that nearly 72% of the nucleotides are CG and about 10% are CpG dinucleotides.

The invention includes determining the methylation status of certain regions of the Markers in a tissue or other biological sample of a subject in which the DNA associated with prostate cancer is amplified and detected. Since a decreased level of the protein encoded by the Marker (i.e., less transcription) is often the result of hypermethylation of a particular region such as the promoter, it is desirable to determine whether such regions are hypermethylated. This is seen most demonstrably in the case of the GSTP1 gene. Hypermethylated regions are those that are methylated to a statistically significant greater degree in samples from diseased tissue as compared to normal tissue.

For purposes of the invention, a nucleic acid probe or reporter specific for certain Marker regions is used to detect the presence of methylated regions of the Marker gene in biological fluids or tissues including prostate tissue, urine, urethral washings, blood, blood components such as serum, ejaculate, and other samples from which prostate proteins could be expected. Oligonucleotide primers based on certain portions of the Marker sequence are particularly useful for amplifying DNA by techniques such as PCR. Any specimen containing a detectable amount of the relevant polynucleotide can be used. Urine and prostate tissue are the preferred samples for determining methylation status. Preferably the sample contains epithelial cells.

Some of the primers/probes or reporter reagents of the invention are used to detect methylation of expression control sequences of the Marker genes. These are nucleic acid sequences that regulate the transcription and, in some cases, translation of the nucleic acid sequence. Thus, expression control sequences can include sequences involved with promoters, enhancers, transcription terminators, start codons (i.e., ATG), splicing signals for introns, maintenance of the correct reading frame of that gene to permit proper translation of the mRNA, and stop codons.

The GSTP1 promoter is an expression control sequence exemplary of a useful Marker. It is a polynucleotide sequence that can direct transcription of the gene to produce a glutathione-s-transferase protein. The promoter region is located upstream, or 5′ to the structural gene. It may include elements which are sufficient to render promoter-dependent gene expression controllable for cell-type specific, tissue-specific, or inducible by external signals or agents; such elements may be located in the 5′ or 3′ regions of the of the polynucleotide sequence.

One method of the invention includes contacting a target cell containing a Marker with a reagent that binds to the nucleic acid. The target cell component is a nucleic acid such as DNA or RNA. The reagents can include probes and primers such as PCR or MSP primers or other molecules configured to amplify and detect the target sequence. For example, the reagents can include priming sequences combined with or bonded to their own reporter segments such as those referred to as Scorpion reagents or Scorpion reporters and described in U.S. Pat. Nos. 6,326,145 and 6,270,967 to Whitcombe et. al. (incorporated herein by reference in their entirety). Though they are not the same, the terms “primers” and “priming sequences” may be used in this specification to refer to molecules or portions of molecules that prime the amplification of nucleic acid sequences.

One sensitive method of detecting methylation patterns involves combining the use of methylation-sensitive enzymes and the polymerase chain reaction (PCR). After digestion of DNA with the enzyme, PCR will amplify from primers flanking the restriction site only if DNA cleavage was prevented by methylation. Exemplary target regions to which PCR primers of the invention are designed include primers which flank the region that lies approximately between −71 and +59 bp according to genomic positioning number of M24485 (Genbank) from the transcription start site of GSTP1.

The method of the invention can also include contacting a nucleic acid-containing specimen with an agent that modifies unmethylated cytosine; amplifying the CpG-containing nucleic acid in the specimen by means of CpG-specific oligonucleotide primers: and detecting the methylated nucleic acid. The preferred modification is the conversion of unmethylated cytosines to another nucleotide that will distinguish the unmethylated from the methylated cytosine. Preferably, the agent modifies unmethylated cytosine to uracil and is sodium bisulfite, however, other agents that modify unmethylated cytosine, but not methylated cytosine can also be used. Sodium bisulfite (NaHSO₃) modification is most preferred and reacts readily with the 5,6-double bond of cytosine, but poorly with methylated cytosine. Cytosine reacts with the bisulfite ion to form a sulfonated cytosine reaction intermediate susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template. Scorpion reporters and reagents and other detection systems similarly distinguish modified from unmodified species treated in this manner.

The primers used in the invention for amplification of a CpG-containing nucleic acid in the specimen, after modification (e.g., with bisulfite), specifically distinguish between untreated DNA, methylated, and non-methylated DNA. In methylation specific PCR (MSPCR), primers or priming sequences for the non-methylated DNA preferably have a T in the 3′ CG pair to distinguish it from the C retained in methylated DNA, and the compliment is designed for the antisense primer. MSP primers or priming sequences for non-methylated DNA usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention are oligonucleotides of sufficient length and appropriate sequence so as to provide specific initiation of polymerization on a significant number of nucleic acids in the polymorphic locus. When exposed to appropriate probes or reporters, the sequences that are amplified reveal methylation status and thus diagnostic information.

Preferred primers are most preferably eight or more deoxyribonucleotides or ribonucleotides capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The priming segment of the primer or priming sequence is preferably single stranded for maximum efficiency in amplification, but may be double stranded. If double stranded, the primer is first treated to separate its strands before being used to prepare extension products. The primer must be sufficiently long to prime the synthesis of extension products in the presence of the inducing agent for polymerization. The exact length of primer will depend on factors such as temperature, buffer, and nucleotide composition. The oligonucleotide primers most preferably contain about 12-20 nucleotides although they may contain more or fewer nucleotides, preferably according to well known design guidelines or rules.

Primers are designed to be substantially complementary to each strand of the genomic locus to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with the 5′ and 3′ flanking sequence(s) to hybridize and permit amplification of the genomic locus.

The primers are employed in the amplification process. That is, reactions (preferably, an enzymatic chain reaction) that produce greater quantities of target locus relative to the number of reaction steps involved. In a most preferred embodiment, the reaction produces exponentially greater quantities of the target locus. Reactions such as these include the PCR reaction. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. The product of the chain reaction is a discrete nucleic acid duplex with termini corresponding to the ends of the specific primers employed.

The primers may be prepared using any suitable method, such as conventional phosphotriester and phosphodiester methods including automated methods. In one such automated embodiment, diethylphosphoramidites are used as starting materials and may be synthesized as described by Beaucage, et at, (Tetrahedron Letters, 22:1859-1862, 1981). A method for synthesizing oligonucleotides on a modified solid support is described in U.S. Pat. No. 4,458,066.

Any nucleic acid specimen, in purified or non-purified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA. The DNA or RNA may be single stranded or double stranded. In the event that RNA is to be used as a template, enzymes, and/or conditions optimal for reverse transcribing the template to DNA would be utilized. In addition, a DNA-RNA hybrid containing one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule so that the specific sequence constitutes the entire nucleic acid.

The nucleic acid-containing specimen used for detection of methylated CpG may be tissue (particularly, prostate tissue and lymphatic tissue), blood or blood components, lymph, urine, urethral washings, ejaculate or other biological samples and may be extracted by a variety of techniques such as that described by Maniatis, et al. (Molecular Cloning: A Laboratory Manual, Cold Spring Harbor, N.Y., pp 280, 281, 1982).

If the extracted sample is impure, it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical or enzymatic means. One physical method of separating nucleic acid strands involves heating the nucleic acid until it is denatured. Typical heat denaturation may involve temperatures ranging from about 80 to 105° C. for up to 10 minutes. Strand separation may also be induced by an enzyme from the class of enzymes known as helicases or by the enzyme RecA, which has helicase activity, and in the presence of riboATP, is known to denature DNA. Reaction conditions that are suitable for strand separation of nucleic acids using helicases are described by Kuhn Hoffmann-Berling (CSH-Quantitative Biology, 43:63, 1978). Techniques for using RecA are reviewed in C. Radding (Ann. Rev. Genetics, 16:405-437, 1982). Refinements of these techniques are now also well known.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. A molar excess (for genomic nucleic acid, usually about 10⁸:1, primer:template) of the two oligonucleotide primers is preferably added to the buffer containing the separated template strands. The amount of complementary strand may not be known if the process of the invention is used for diagnostic applications, so the amount of primer relative to the amount of complementary strand cannot always be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90-100° C. for up to 10 minutes, preferably from 1 to 4 minutes. After this heating period, the solution is allowed to cool to room temperature, which is preferable for the primer hybridization. To the cooled mixture is added an appropriate agent for effecting the primer extension reaction (the “agent for polymerization”), and the reaction is allowed to occur under conditions known in the art. The agent for polymerization may also be added together with the other reagents if it is heat stable. This synthesis (or amplification) reaction may occur at room temperature up to a temperature at which the agent for polymerization no longer functions.

The agent for polymerization may be any compound or system that will function to accomplish the synthesis of primer extension products, preferably enzymes. Suitable enzymes for this purpose include, for example, E. coli DNA polymerase 1. Klenow fragment of E. coli DNA polymerase I, T4 DNA polymerase, other available DNA polymerases, polymerase mutants, reverse transcriptase, and other enzymes, including heat-stable enzymes (e.g., those enzymes which perform primer extension after being subjected to temperatures sufficiently elevated to cause denaturating). A preferred agent is Taq polymerase. Suitable enzymes will facilitate combination of the nucleotides in the proper manner to form the primer extension products complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Most preferably, the method of amplifying is by PCR. Alternative methods of amplification can also be employed as long as the methylated and non-methylated loci amplified by PCR using the primers of the invention is similarly amplified by the alternative means.

The amplified products are preferably identified as methylated or non-methylated with a probe or reporter specific to the product as described in U.S. Pat. No. 4,683,195 to Mullis et. al., incorporated herein by reference in its entirety. Advances in the field of probes and reporters for detecting polynucleotides are well known to those skilled in the art. Optionally, the methylation pattern of the nucleic acid can be confirmed by other techniques such as restriction enzyme digestion and Southern blot analysis. Examples of methylation sensitive restriction endonucleases which can be used to detect 5′CpG methylation include SmaI, SacII, EagI, MspI, HpaII, BstUI and BssHII.

In another aspect of the invention a methylation ratio is used. This can be done by establishing a ratio between the amount of amplified methylated species of Marker attained and the amount of amplified reference Marker or non-methylated Marker region amplified. This is best done using quantitative real-time PCR. Ratios above an established or predetermined cutoff or threshold are considered hypermethylated and indicative of having a proliferative disorder such as cancer (prostate cancer in the case of GSTP1). Cutoffs are established according to known methods in which such methods are used for at least two sets of samples: those with known diseased conditions and those with known normal conditions. The reference Markers of the invention can also be used as internal controls. The reference Marker is preferably a gene that is constitutively expressed in the cells of the samples such as Beta Actin. Established or predetermined values (cutoff or threshold values) are also established and used in methods according to the invention in which a ratio is not used. In this case, the cutoff value is established with respect to the amount or degree of methylation relative to some baseline value such as the amount or degree of methylation in normal samples or in samples in which the cancer is clinically insignificant (is known not to progress to clinically relevant states or is not aggressive). These cutoffs are established according to well-known methods as in the case of their use in methods based on a methylation ratio.

The inventive methods and kits can include steps and reagents for multiplexing. That is, more than one Marker can be assayed at a time.

Since a decreased level of transcription of the gene associated with the Marker is often the result of hypermethylation of the polynucleotide sequence and/or particular elements of the expression control sequences (e.g., the promoter sequence), primers prepared to match those sequences were prepared. Accordingly, the invention provides methods of detecting or diagnosing a cell proliferative disorder by detecting methylation of particular areas within the expression control or promoter region of the Markers. Probes useful for detecting methylation of these areas are useful in such diagnostic or prognostic methods. Preferred molecules for the detection of Markers are shown below. The short name for the Marker gene is shown in parentheses along with the type of detection system employed. Antisense only refers to the orientation of the primer that is so designated in relationship to the priming sequence of the other member of the pair with which it is associated. It is not necessarily antisense with respect to genomic DNA.

SEQ ID NO. 1 (GSTP1 SCORPION):
CCCCGAACGTCGACCGCTCGGGG-BHQ-HEG-CGATTTCGGG
GATTTTAGGGCGT
SEQ ID NO. 2 (GSTP1 SCORPION Antisense Primer):
AAAATCCCGCGAACTCCCGCC
SEQ ID NO. 3 (GSTP1 SCORPION):
CCCGAACGTCGACCGCTTTCGGG-BHQ-HEG-CGATTTCGGG
GATTTTAGGGCGT
SEQ ID NO. 4 (GSTP1 SCORPION Antisense Primer):
AAAATCCCGCGAACTCCCGCC
SEQ ID NO. 5 (GSTP1 SCORPION):
CGGCGGGAGTTCGCGGGCGCCG-BHQ-HEG-ACTAAATCACG
ACGCCGACCGC
SEQ ID NO. 6 (GSTP1 SCORPION Antisense Primer):
CGGTTAGTTGCGCGGCGATTTC
SEQ ID NO. 7 (GSTP1 SCORPION):
CGGGAGTTCGCGGGTCCCG-BHQ-HEG-ACTAAATCACGACGC
CGACCGC
SEQ ID NO. 8 (GSTP1 SCORPION Antisense Primer)
CGGTTAGTTGCGCGGCGATTTC
SEQ ID NO. 9 (GSTP1 SCORPION):
GTGGTTGATGTTTGGGGTATCAACCAC-BHQ-HEG_AATCCCAC
AAACTCCCACCAACC
SEQ ID NO. 10 (GSTP1 SCORPION Antisense Primer):
GTGGTGATTTTGGGGATTTTAGGGTGT
SEQ ID NO. 11 (GSTP1 SCORPION):
ACCCCAGTGGTTGATGTTTGGGGT-BHQ-HEG-AATCCCACAAA
CTCCCACCAACC
SEQ ID NO. 12 (GSTP1 SCORPION Antisense Primer):
GTGGTGATTTTGGGGATTTTAGGGTGT
SEQ ID NO. 13 (GSTP1 SCORPION):
CCCCACAGGTTGGTGGGAGTTTGTGGGG-BHQ-HEG-
CCCAATACTAAATCACAACACCAACCAC
SEQ ID NO. 14 (GSTP1 SCORPION Antisense Primer):
TGGTTAGTTGTGTGGTGATTTTGGGGA
SEQ ID NO. 15 (GSTP1 SCORPION):
CCCCGAACGTCGACCGCTCGGGG-BHQ-HEG-CGATTTCGGGGA
TTTTAGGGCGT
SEQ ID NO. 16 (GSTP1 SCORPION Antisense Primer):
AAAATCCCGCGAACTCCCGCC
SEQ ID NO. 17 (GSTP1 SCORPION):
CGCACGCCGAACGTCGACCGCAAACGTGCG-BHQ-HEG-
CGATTTCGGGGATTTTAGGGCGT
SEQ ID NO. 18 (GSTP1 SCORPION Antisense Primer):
AAAATCCCGCGAACTCCCGCC
SEQ ID NO. 19 (GSTP1 SCORPION):
CGCACGGCGAACTCCCGCCGACGTGCG BHQ-HEG-TGTAGCGG
TCGTCGGGGTTG
SEQ ID NO. 20 (GSTP1 SCORPION Antisense Primer):
GCCCCAATACTAAATCACGACG
SEQ ID NO. 21 (GSTP1 SCORPION):
CCGACGCACAAAAAAACACCCTAAAATCCGTCGG-BHQ-HEG-
GGTTAGTTGTGTGGTGATTTT
SEQ ID NO. 22 (GSTP1 SCORPION Antisense Primer):
CACAACACCAACCACTCTTC
SEQ ID NO. 23 (GSTP1 TAQMAN PRIMER):
CGTGATTTAGTATTGGGGCGGAGCGGGGC
SEQ ID NO. 24 (GSTP1 TAQMAN PRIMER):
ATCCCCGAAAAACGAACCGCGCGTA
SEQ ID NO. 25 (GSTP1 TAQMAN PROBE):
TCGGAGGTCGCGAGGTTTTCGTTGGA
SEQ ID NO. 26 (GSTP1 SCORPION):
CGGCCCTAAAACCGCTACGAGGGCCG-BHQ-HEG-GAAGCGGGTGT
GTAAGTTTCGG
SEQ ID NO. 27 (GSTP1 SCORPION Antisense Primer):
ACGAAATATACGCAACGAACTAACGC
SEQ ID NO. 28 (GSTP1 SCORPION):
CCGGTCGCGAGGTTTTCGACCGG-BHQ-HEG-CCGAAAAACGAA
CCGCGCGTA
SEQ ID NO. 29 (GSTP1 SCORPION Antisense Primer):
GGGCGGGATTATTTTTATAAGGTTCGG
SEQ ID NO. 30 (RASSF1A SCORPION):
GCCGCGGTTTCGTTCGGTTCGCGGC-BHQ-HEG-CCCGTACTTC
GCTAACTTTAAACG
SEQ ID NO. 31 (RASSF1A SCORPION Antisense Primer):
GCGTTGAAGTCGGGGTTC
SEQ ID NO. 32 (RARB2 SCORPION):
CGGCGCCCGACGATACCCAAAGCGCCG-BHQ-HEG-AACGCGAG
CGATTCGAGTAG
SEQ ID NO. 33 (RARB2 SCORPION Antisense Primer):
CTTACAAAAAACCTTCCGAATACG
SEQ ID NO. 34 (APC SCORPION):
GCCGGCGGGTTTTCGACGGGCCGGC-BHQ-HEG-CGAACCAAA
ACGCTCCCCA
SEQ ID NO. 35 (APC SCORPION Antisense Primer):
GTCGGTTACGTGCGTTTATATTTAG
SEQ ID NO. 36 (ACTIN SCORPION):
GCGCCCAACCGCACAAGGGCGC-BHQ-HEG-GGGTATATTT
TCGAGGGGTACG
SEQ ID NO. 37 (ACTIN SCORPION Antisense Primer):
CGACCCGCACTCCGCAAT
SEQ ID NO. 38(ACTIN SCORPION):
CCGCGCATCACCACCCCACACGCGCGG-BHQ-HEG-GGAGTAT
ATAGGTTGGGGAAGTTTG
SEQ ID NO. 39 (ACTIN SCORPION Antisense Primer):
AACACACAATAACAAACACAAATTCAC
SEQ ID NO. 40 (ACTIN SCORPION):
CCCGGCTAAACCCACCATCCAGCCGGG-BHQ-HEG-GGGAGG
GTAGTTTAGTTGTGGTT
SEQ ID NO. 41 (ACTIN SCORPION Antisense Primer):
CAAAACAAAAAAACTAAATCTACACAACC
SEQ ID NO. 42 (ACTIN SCORPION):
CCGCGGAACATTCAACTCAACCGCGG-BHQ-HEG-GGAGGAGGA
AGGTAGGTTTTT
SEQ ID NO. 43 (ACTIN SCORPION Antisense Primer):
ACATACAACAATCAATAACATAAAACCAC
SEQ ID NO. 44 (PTGS2/COX2 SCORPION):
CACGCCGCCGTATCTAGGCGTG-BHQ-HEG-GTTTGTTTCGACGT
GATTTTTTCGA
SEQ ID NO. 45 (PTGS2/COX2 Antisense Primer):
GCAAAAAATCCCCTCTCCCGC
SEQ ID NO. 46 (PTGS2/COX2 SCORPION):
GCCGCGCACAAATTTCCGCGGC-BHQ-HEG-GAATTGGTTTTC
GGAAGCGTTCG
SEQ ID NO. 47 (PTGS2/COX2 Antisense Primer):
CCCGAATTCCACCGCC
SEQ ID NO. 48 (PTGS2/COX2 SCORPION):
GGCGGAACGCACAAATTTCCGCC-BHQ-HEG-GAATTGGTTTTC
GGAAGCGTTCG
SEQ ID NO. 49 (PTGS2/COX2 Antisense Primer):
CCCGAATTCCACCGCC
SEQ ID NO. 50 (PTGS2/COX2 SCORPION):
TGCCGCCGCCGTATCTAATGGCGGCA-BHQ-HEG-GTTT
GTTTCGACGTGATTTTTTCGA
SEQ ID NO. 51 (PTGS2/COX2 Antisense Primer):
GCAAAAAATCCCCTCTCCCGC
SEQ ID NO. 52 (CDH1 SCORPION):
CGCCGAATACGATCGGCG-BHQ-HEG-GTTCGTTTTAGTTCC
TGTTCGA
SEQ ID NO. 53 (CDH1 SCORPION Antisense Primer):
ACCGAAAACGCCGAACGA
SEQ ID NO. 54 (15LO1 SCORPION):
GGCGGCGTTCGGGCCGCC-HEG-BHQ-CCGTACGAACCACAATCGC
SEQ ID NO. 55 (15LO1 SCORPION Antisense Primer):
GGGGTTTCGTTTTATGTCGGT
BHQ = Black Hole Quencher (BioSearch Technologies, San Fransisco, CA)
HEG = Hexaethylene glycol

The kits of the invention can be configured with a variety of components provided that they all contain at least one primer or probe or a detection molecule (e.g., Scorpion reporter). In one embodiment, the kit includes reagents for amplifying and detecting hypermethylated Marker segments. Optionally, the kit includes sample preparation reagents and/or articles (e.g., tubes) to extract nucleic acids from samples.

In a preferred kit, reagents necessary for one-tube MSP are included such as, a corresponding PCR primer set, a thermostable DNA polymerase, such as Taq polymerase, and a suitable detection reagent(s) such as hydrolysis probe or molecular beacon. In optionally preferred kits, detection reagents are Scorpion reporters or reagents. A single dye primer or a fluorescent dye specific to double-stranded DNA such as ethidium bromide can also be used. The primers are preferably in quantities that yield high concentrations. Additional materials in the kit may include: suitable reaction tubes or vials, a barrier composition, typically a wax bead, optionally including magnesium; necessary buffers and reagents such as dNTPs; control nucleic acid (s) and/or any additional buffers, compounds, co-factors, ionic constituents, proteins and enzymes, polymers, and the like that may be used in MSP reactions. Optionally, the kits include nucleic acid extraction reagents and materials.

In a most preferred kit of the invention, instructions to conduct the assay on patients with prostate samples assessed as having a Gleason score of 7 or higher are provided. In another kit according to the invention, the instructions are to conduct the assay on patients with samples assessed as having a Gleason score greater than 7. In another kit according the invention, instructions are provided to conduct the assay on patients with a PSA level greater than 2.5 ng/ml and in another kit the instructions are provided to conduct the assay on patients with PSA levels of 2-4 ng/ml. The instructions may also indicate that a positive methylation result should be followed up with a biopsy.

### EXAMPLES

### Example 1

### Methylation Testing and Gleason Score

Prostate samples were obtained from patients with known clinical outcomes. Gleason scores were assigned to the samples according to well-known methods. From these samples, 52 were found to have Gleason scores less than 7, 36 had Gleason scores of 7, and 12 had Gleason scores greater than 7.

Methylation assays were conducted on each set using GSTP1 (Seq ID No 19, 20) and APC reagents (Seq ID No 34, 35).

The methylation assays were conducted as follows. Genomic DNA was modified using a commercially available sodium bisulfite conversion reagent kit (Zymo Research, Orange, Calif., USA). This treatment converted all Cytosines in unmethylated DNA into Uracil, whereas in methylated DNA only cytosines not preceding guanine were converted into Uracil. All cytosines preceeding guanine (in a CpG dinucletide) remained as cytosine.

Sodium bisulfite modified genomic DNA (100-150 ng) was amplified in a 25 μl reaction containing the following components: 67 mM Tris pH 8.8, 16.6 mM (NH₄)₂SO₄, 6.7 mM MgCl₂, 10 mM beta mercaptoethanol; 1.25 mM each dATP, dCTP, dGTP, dTTP, 1 U Hot start Taq DNA Polymerase, 250 nM Scorpion probe. 250 nM reverse or forward primer (depending on scorpion design), 625 nM of passive reference dye.

The samples were then tested in a quantitative real-time PCR assay on the Cepheid SmartCycler® PCR instrument. The PCR conditions used were:
95° C. for 60 sec; then 40 cycles of 95° C. for 30 sec. 59° C. for 30 sec, and a final extension at 72° C. for 5 min. Optical data was collected at 59° C. for every cycle.

A methylation ratio [(copy # of Marker/copy# of B-actin)×1000] cutoff of 1 was established for GSTP1 and a methylation ratio cutoff of 10 was established for APC. The cutoffs were based on clinically relevant sensitivity and specificity requirements. Results were as shown in the following tables:

TABLE 1
GSTP1

| Gleason | No. | | Not- | Undeter- | |
|---|---|---|---|---|---|
| Score | Samples | Methylated | Methylated | mined | Sensitivity |
| <7 | 52 | 30 | 16 | 6 | 57.6 |
| 7 | 36 | 24 | 8 | 4 | 66.6 |
| >7 | 12 | 11 | 1 | 0 | 91.6 |

TABLE 2
APC

| Gleason | No. | | Not- | Undeter- | |
|---|---|---|---|---|---|
| Score | Samples | Methylated | Methylated | mined | Sensitivity |
| <7 | 52 | 33 | 12 | 7 | 63.46 |
| 7 | 36 | 25 | 8 | 3 | 69.44 |
| >7 | 12 | 11 | 1 | 0 | 91.6 |

These results show that the methylation assay provides accurate information about the prostate cancer status of patients with Gleason scores above 7. Useful and relatively accurate information is also provided in patients with Gleason scores of 7, particularly when combined with other diagnostic or prognostic information.

There is currently a large dichotomy in the Gleason 6 and 7 populations. Approximately half of these patients have a poor prognosis and half have a good prognosis. Until now, there has been no way to determine who will benefit from more aggressive treatment and who will not. The higher sensitivity of methylation assays in cancers with a Gleason score >7, typically the more aggressive cancers, enables one to predict that a patient with a methylation assay result above the cutoff will have a poor prognosis as a result of an aggressive cancer. The methylation data above would predict that 66-69% of the Gleason 7 patients will have a poor prognosis and should be considered for aggressive treatment while the remaining on-third could go into watchful waiting. Thus, the strong correlation of the positivity in the methylation assay in the Gleason score>7 population (the poor prognosis population) indicates prognostic as well as diagnostic value.

### Example 2

### Serum Assay

Serum samples were obtained from patients with known prostate cancer outcomes and from whom biopsy samples were taken and Gleason scores adduced. Among these samples, 55 were from patients with no cancer, 36 were from patients with Gleason scores of 5-6, and 21 were from patients with Gleason scores of 7-8.

Methylation status was determined according to the method of Example 1.

The GSTP1 Marker correctly detected methylation in 26% the samples from patients with a Gleason score of 7-8 and did not detect methylation in those patients with Gleason scores of 5-6 or who were non-cancerous. The APC Marker correctly detected methylation in 26% of the samples from patients with a Gleason score of 7-8, in up to 9 instances it also detected methylation in patients with a Gleason score of 5-6 or who were non-cancerous. The combined specificity of the two Markers was 84% and sensitivity was 18% with a Gleason score of 5-6 and 38% with a Gleason score of 7-8.

A third and fourth Marker, RASSF1a and RARb2 were then added to the group of Markers used to detect methylation to yield a specificity of 82%, a sensitivity of 25% for Gleason scores of 5-6 and 58% for Gleason scores of 7-8. Thus, the inclusion of additional or different methylation markers can be used to boost sensitivity where serum testing is desired and both sensitivity and specificity requirements are heightened.

Additional Marker testing data is shown and described below.

There were 58 samples including 34 prostate adenocarcinoma (CaP), 24 Prostate Benign (Neg), 6 HG-PIN (Neg), 2 Atrophy (Neg), 4 Atypia (Neg), and 2 Inflamatory (Neg). Three samples were missing a biopsy report and one sample failed test (no Actin-hskg Ct value). Markers for GSTP1, RASSF1, RARB2, APC, CDH1 and 15-LO-1 were used.

Reagents were prepared for the msPCR assays using these Markers are shown in Table 3.

TABLE 3

| Reagents | Amount (ul) | Final Conc. | |
|---|---|---|---|
| DNA (ul) | 5.0 | — | |
| 10x Roche Buffer (no MgCl) | 2.5 | 1x | |
| Faststart Taq 5 U/ul | 0.2 | 0.04 | U |
| 6.25 uM probe -Primer mix | 1 | 0.25 | uM |
| 25 mM dNTPs | 1.25 | l.25 | mM |
| 1 mM Rox (l:500dilution) | 1 | 80 | nM |
| MgCl2 (25 mM) | 6.7 | 6.7 | mM |

| | | |
|---|---|---|
| Total reaction | 25.0 | — |

Using 0.15 uM final probe-primer concentration for 3 GSTP1 mixtures.
Primer/Probes for the Markers were as follows.
GSTP1: Seq ID No. 26/27; Seq ID No. 28/29; Seq ID No. 19/20
RASSF1: Seq ID No. 30/31
RARB2: Seq ID No. 32/33
APC: Seq ID No. 34/35
CDH1: Seq ID No. 52/53
15-LO-1: Seq ID No. 54/55
Beta Actin: Seq ID No. 38/39
PCR conditions are shown in Table 4:

TABLE 4

| | | |
|---|---|---|
| Parameters | Time | Cycles |

| | | | |
|---|---|---|---|
| 95 C. | 5 | min | 1 |
| 95 C. | 30 | sec | 55 |
| 59 C. | 30 | sec | (Optics-on) |
| 72 C. | 30 | sec | |
| 72 C. | 5 | min | 1 |

Table 5 shows the Ct values with six gene specific markers and one hkg and includes available information of Gleason Score and PAS for 58 samples.

TABLE 5

| Sample | ID | Actin | APC | GSTP1 | Rass | RARb | CDH1 | 15_LO | GS (R/L) | PSA 9 ng/ml) |
|---|---|---|---|---|---|---|---|---|---|---|
| Cap | 5 | 27.1 | 35.3 | | | 38.8 | 36.9 | | 7/7 | 10 |
| Cap | 6 | 28.5 | 38.9 | 49.7 | 36.8 | | | | 5/5 | 1 |
| Cap | 8 | 29.8 | | | | | | | 7/6 | 11 |
| Cap | 9 | 28.7 | 37.1 | | | | 48.7 | | 6/0 | 5 |
| Cap | 10 | 29.9 | | 35.8 | | | | 38.5 | 8/9 | 135 |
| Cap | 12 | 24.4 | 37.7 | | | 38.3 | 34.6 | | 0/6-7 | N/A |
| Cap | 13 | 26.4 | 39.4 | | | | 48.1 | | 6/7 | N/A |
| Cap | 15 | 26.6 | 39.6 | | | | 53.9 | | 4/0 | N/A |
| Cap | 16 | 28.7 | 42.7 | 48.5 | | | 40.6 | 40.0 | 0/5 | N/A |
| Cap | 18 | 26.7 | 38.6 | | | | | | 0/7 | N/A |
| Cap | 20 | 26.2 | 38.7 | | | | 40.8 | | N/A | N/A |
| Cap | 22 | 27.4 | 35.3 | | | | | | 6/0 | N/A |
| Cap | 23 | 23.9 | 34.7 | | 37.0 | | | | 0/8 | N/A |
| Cap | 26 | 25.7 | | 40.2 | | | | | 0/6 | N/A |
| Cap | 27 | 26.4 | 36.0 | 31.9 | | | | | N/A | N/A |
| Cap | 32 | 25.1 | 38.8 | 32.6 | | | | | 7/7 | N/A |
| Cap | 1S8LMSB | 28.4 | 34.7 | | | | | | N/A | N/A |
| Cap | AH11BSA | 22.5 | 34.3 | | 38.5 | | | 37.4 | N/A | N/A |
| Cap | SB6JDSC | 23.9 | 41.6 | | | | 41.1 | | N/A | N/A |
| Cap | VGKJASA | 23.2 | | 39.2 | | | | | N/A | N/A |
| Cap | WH24ESB | 25.7 | 36.3 | | | | 42.4 | | N/A | N/A |
| Cap | Y3IG8SC | 22.3 | 36.5 | | | | | | N/A | N/A |
| Cap | 5091 | 26.0 | | | 52.5 | | | | 6 | 0.9 |
| Cap | 5098 | 26.1 | 36.7 | | 49.4 | | | | 6 | 7.4 |
| Cap | 5108 | 23.7 | 37.5 | | | | | | 7 | 2.2 |
| Cap | 5113 | 25.9 | | 40.7 | | | | | 6 | 11.8 |
| Cap | 5115 | 25.9 | 34.8 | | 39.4 | | 43.1 | | 7 | 5.1 |
| Cap | 5129 | 30.4 | | | | | | | 7 | 3.3 |
| Cap | 5133 | 24.2 | 33.6 | | | | | | 6 | 7.4 |
| Cap | 5134 | 26.0 | 36.5 | 51.4 | 52.0 | | | | 6 | 6.2 |
| Cap | 5333 | 27.5 | 40.3 | | 37.4 | | | | 7 | 8.7 |
| Cap | 5343 | 29.5 | | | 48.9 | | 54.6 | | 8 | 7.9 |
| Cap | 5349 | 35.6 | | 41.7 | 49.0 | | | | 6 | 6.8 |
| Cap | 5354 | 28.4 | 34.7 | | | 33.6 | | | 6 | 3.6 |
| HG-PIN | 7 | 27.6 | | 37.9 | | | | | | 9 |
| HG-PIN | 2810 | 29.0 | 38.4 | | 41.0 | | | | | 5.7 |
| HG-PIN | 3002 | 26.9 | 35.8 | 54.9 | | | | | | N/A |
| HG-PIN | 3210 | 25.5 | 37.8 | 39.1 | 44.6 | | 54.8 | | | N/A |
| HG-PIN | 3319 | 23.7 | 37.8 | 47.6 | | | | | | N/A |
| HG-PIN | 4079 | 28.8 | 36.4 | 47.8 | | | | | | 2.2 |
| Benign | 11 | 29.5 | 50.3 | | | | 51.9 | | | N/A |
| Benign | 14 | 31.9 | | | | | 44.1 | | | N/A |
| Benign | 21 | 28.3 | 49.4 | | | | | | | N/A |
| Benign | 3263 | 24.9 | | | | 41.0 | | | | 1.4 |
| Benign | 3602 | 26.0 | 36.2 | 44.3 | 49.0 | | | | | 10.1 |
| Benign | 3836 | 25.3 | 38.3 | | | | | | | N/A |
| Benign | 3882 | 28.3 | 36.2 | 45.9 | | | | | | N/A |
| Benign | 4017 | 27.6 | | | | | | | | 7.3 |
| Benign | 5569 | 28.9 | 28.7 | | 39.3 | | | | | 3.0 |
| Atrophy | 3006 | 27.5 | 39.1 | 47.8 | | 40.9 | | | | N/A |
| Atrophy | 3285 | 26.1 | 38.7 | | | | | | | N/A |
| Atypia | 3358 | 23.5 | | | | 41.9 | 39.1 | | | 2.8 |
| Atypia | 3512 | 26.9 | 37.4 | | | | 48.4 | | | 5.0 |
| Atypia | 3804 | 27.4 | | | | | | 42.4 | | 3.9 |
| Atypia | 4393 | 28.9 | 37.4 | | 48.4 | | | | | 3.8 |
| Inflam | 17 | 29.4 | 38.0 | | 45.7 | | | | | N/A |
| Inflam | 2989 | 29.2 | 38.1 | | 40.0 | | | | | N/A |
| Inflam | 3182 | 25.3 | 37.3 | | | | 45.7 | | | 7.2 |

Blank - not determined for Ct after 55 cycles of QMSP.

Sensitivity and specificity were determined directly by Ct values shown in Table 6.

TABLE 6

| Ct cutoff setting | | | | | | |
|---|---|---|---|---|---|---|
| for 6 or 4 markers | APC | GSTP1 | Rass | RARb2 | CDH1 | 15_LO |
| Sensitivity 55% | 37 | 37 | 40 | 40 | 40 | 40 |
| Specificity 82% | | | | | | |
| Sensitivity 52% | 37 | 37 | 40 | 40 | not used | not used |
| Specificity 84% | | | | | | |
| Sensitivity 39% | 36 | 37 | 39 | 40 | not used | not used |
| Specificity 95% | | | | | | |
| Sensitivity 37% | 35 | 37 | 39 | 40 | not used | not used |
| Specificity 97% | | | | | | |

### Example 3

### Urine Assay

Urine samples were obtained from patients with known prostate cancer outcomes and from whom biopsy samples were taken and Gleason scores adduced. Among these samples, 42 were from patients with Gleason scores of 4-6 and 10 were from patients with Gleason scores of 7-9.

Methylation status was determined according to the method of Example 1 using the Cepheid Smart Cycler PCR instrument.

The combined specificity of the two Markers, GSTP1 and RARb2 was 89% for post-massage urine samples and 91% for post biopsy samples. Methylation assays with post massage samples were 40% sensitive in those with Gleason scores below 7 and 78% for those with scores greater than 7. Thus, noninvasive sampling can be used in conjunction with the other aspects of the invention.

### Example 4

### Serum Assay with PSA Result (Prophetic)

Serum samples are obtained from patients with known prostate cancer outcomes. PSA concentrations are determined according to standard clinical methods. Among these samples, 55 are from patients with no cancer having PSA levels of 1-2 ng/ml, 36 are from patients with PSA levels of 2-4 ng/ml, and 21 are from patients with PSA levels greater than 4. Patients with PSA levels greater than 4 are indicated for biopsies according to well-established clinical guidelines.

The methylation status for patients with PSA levels below 4 are determined according to the method of Example 1.

The GSTP1 Marker detects methylation in 20% the samples from patients with a PSA level of 2-4. These patients are biopsied and found to have a Gleason score of 7 or greater. Further treatment is likely indicated in these patients. Hypermethylation is not found in any samples from patients with a PSA value less than 2. APC, RASSF1A, 15-LO-1, and CDH1 Markers are used in a separate methylation assays of these patients and 15% of the samples are found to be hypermethylated. These patients are biopsied and found to have a Gleason score of 7 or greater. Further treatment is likely indicated in these patients. The combined specificity of two Markers is 95% and sensitivity is 85% in patients with PSA levels below 4.

A patient with a PSA score that makes the need for biopsy uncertain is stratified according to the outcome of a methylation assay. This can be particularly useful in a watchful-waiting course of therapy or in a therapy monitoring strategy in general. The patient is periodically tested with a non-biopsy assay such as the PSA test and tested for DNA methylation status of prostate Markers when results that would indicate biopsy are ambiguous or difficult to interpret. A methylation result greater than a pre-determined cutoff indicates a biopsy is necessary and that a Gleason score of 7 or greater is likely to be at least one result of such biopsy.

We claim:

1. A method of predicting the recurrence or aggressiveness of prostate cancer comprising, a) determining the Gleason score of a prostate sample, and b) determining the methylation status of a Marker in a biological sample for those patients having a Gleason score of 7 or greater; wherein methylation that exceeds a pre-determined value is indicative of an aggressive or recurrent cancer and methylation that does not exceed such pre-determined value is indicative of indolent cancer.

2. The method according to claim 1 further comprising measuring the presence of a reference Marker.

3. The method according to claim 2 wherein the reference Marker is selected from the group consisting of beta Actin and PTGS2.

4. The method of claim I wherein a combination of Markers is assayed and includes
a Marker for GSTPI and a Marker for APC, RASSFI A, 15-LO-I, or CDHI.

5. The method of claim I wherein the sample from which methylation status is determined is urine, urethral washing, blood, a blood component, ejaculate, or circulating cells.

6. The method of claim 1 wherein said sample is serum or plasma.

7. A kit for conducting an assay to predict the course or aggressiveness of prostate cancer, comprising: nucleic acid amplification and detection reagents and instructions that direct its use in patients in whom a Gleason score of 7 or higher was adduced.

8. The kit of claim 7 wherein the reagents include a member of the group consisting of Seq. ID No. 26 and 27.

9. The kit of claim 8 wherein the PCR priming reagents consist essentially of of Seq. ID No. 26 and 27.

10. The kit of claim 7 wherein the reagents include a member of the group consisting of Seq. ID No. 28 and 29.

11. The kit of claim 7 wherein the reagents include a member of the group consisting of Seq. ID No. 32 and 33.

12. The kit of claim 7 wherein the reagents include a member of the group consisting of Seq. ID No. 52 and 53.

13. The kit of claim 7 wherein the reagents include a member of the group consisting of Seq. ID No. 54 and 55.1

14. The kit of claim 7 wherein the reagents detect the hypermethylation of a gene selected from the group consisting of GSTP1, APC, RASSF1A, 15-LO-1, and CDH1.

15. The method of claim 1 further comprising establishing a methylation ratio and determining whether the methylation ratio exceeds a cutoff value.

16-20. (canceled)


SEQUENCE LISTING (69 sequences)

SEQ ID NO: 1 - DNA, 46 bp, Artificial Sequence
  Description of Artificial Sequence Synthetic GSTP1 scorpion primer
ccccgaacgtcgaccgctcggggcgatttcggggattttagggcgt

SEQ ID NO: 2 - DNA, 21 bp, Artificial Sequence
  Description of Artificial Sequence Synthetic GSTP1 scorpion antisense primer
aaaatcccgcgaactcccgcc

SEQ ID NO: 3 - DNA, 46 bp, Artificial Sequence
  Description of Artificial Sequence Synthetic GSTP1 scorpion primer
cccgaacgtcgaccgctttcgggcgatttcggggattttagggcgt

SEQ ID NO: 4 - DNA, 21 bp, Artificial Sequence
  Description of Artificial Sequence Synthetic GSTP1 scorpion antisense primer
aaaatcccgcgaactcccgcc

SEQ ID NO: 5 - DNA, 44 bp, Artificial Sequence
  Description of Artificial Sequence Synthetic GSTP1 scorpion primer
cggcgggagttcgcgggcgccgactaaatcacgacgccgaccgc

SEQ ID NO: 6 - DNA, 22 bp, Artificial Sequence
  Description of Artificial Sequence Synthetic GSTP1 scorpion antisense primer
cggttagttgcgcggcgatttc

SEQ ID NO: 7 - DNA, 41 bp, Artificial Sequence
  Description of Artificial Sequence Synthetic GSTP1 scorpion primer
cgggagttcgcgggtcccgactaaatcacgacgccgaccgc

SEQ ID NO: 8 - DNA, 22 bp, Artificial Sequence
  Description of Artificial Sequence Synthetic GSTP1 scorpion antisense primer
cggttagttgcgcggcgatttc

SEQ ID NO: 9 - DNA, 50 bp, Artificial Sequence
  Description of Artificial Sequence Synthetic GSTP1 scorpion primer
gtggttgatgtttggggtatcaaccacaatcccacaaactcccaccaacc

SEQ ID NO: 10 - DNA, 27 bp, Artificial Sequence
  Description of Artificial Sequence Synthetic GSTP1 scorpion antisense primer
gtggtgattttggggattttagggtgt

SEQ ID NO: 11 - DNA, 47 bp, Artificial Sequence
  Description of Artificial Sequence Synthetic GSTP1 scorpion primer
accccagtggttgatgtttggggtaatcccacaaactcccaccaacc

SEQ ID NO: 12 - DNA, 27 bp, Artificial Sequence
  Description of Artificial Sequence Synthetic GSTP1 scorpion primer
gtggtgattttggggattttagggtgt

SEQ ID NO: 13 - DNA, 56 bp, Artificial Sequence
  Description of Artificial Sequence Synthetic GSTP1 scorpion primer
ccccacaggttggtgggagtttgtggggcccaatactaaatcacaacaccaaccac

SEQ ID NO: 14 - DNA, 27 bp, Artificial Sequence
  Description of Artificial Sequence Synthetic GSTP1 scorpion antisense primer
tggttagttgtgtggtgattttgggga

SEQ ID NO: 15 - DNA, 46 bp, Artificial Sequence; identical to SEQ ID NO: 1
  Description of Artificial Sequence Synthetic GSTP1 scorpion primer

SEQ ID NO: 16 - DNA, 21 bp, Artificial Sequence
  Description of Artificial Sequence Synthetic GSTP1 scorpion antisense primer
aaaatcccgcgaactcccgcc

SEQ ID NO: 17 - DNA, 53 bp, Artificial Sequence
  Description of Artificial Sequence Synthetic GSTP1 scorpion primer
cgcacgccgaacgtcgaccgcaaacgtgcgcgatttcggggattttagggcgt

SEQ ID NO: 18 - DNA, 21 bp, Artificial Sequence
  Description of Artificial Sequence Synthetic GSTP1 scorpion antisense primer
aaaatcccgcgaactcccgcc

SEQ ID NO: 19 - DNA, 47 bp, Artificial Sequence
  Description of Artificial Sequence Synthetic GSTP1 scorpion primer
cgcacggcgaactcccgccgacgtgcgtgtagcggtcgtcggggttg

SEQ ID NO: 20 - DNA, 22 bp, Artificial Sequence
  Description of Artificial Sequence Synthetic GSTP1 scorpion antisense primer
gccccaatactaaatcacgacg

SEQ ID NO: 21 - DNA, 55 bp, Artificial Sequence
  Description of Artificial Sequence Synthetic GSTP1 scorpion primer
ccgacgcacaaaaaaacaccctaaaatccgtcggggttagttgtgtggtgatttt

SEQ ID NO: 22 - DNA, 20 bp, Artificial Sequence
  Description of Artificial Sequence Synthetic GSTP1 scorpion antisense primer
cacaacaccaaccactcttc

SEQ ID NO: 23 - DNA, 29 bp, Artificial Sequence
  Description of Artificial Sequence Synthetic GSTP1 Taqman primer
cgtgatttagtattggggcggagcggggc

SEQ ID NO: 24 - DNA, 25 bp, Artificial Sequence
  Description of Artificial Sequence Synthetic GSTP1 Taqman primer
atccccgaaaaacgaaccgcgcgta

SEQ ID NO: 25 - DNA, 26 bp, Artificial Sequence
  Description of Artificial Sequence Synthetic GSTP1 Taqman probe
tcggaggtcgcgaggttttcgttgga

SEQ ID NO: 26 - DNA, 48 bp, Artificial Sequence
  Description of Artificial Sequence Synthetic GSTP1 scorpion primer
cggccctaaaaccgctacgagggccggaagcgggtgtgtaagtttcgg

SEQ ID NO: 27 - DNA, 26 bp, Artificial Sequence
  Description of Artificial Sequence Synthetic GSTP1 scorpion antisense primer
acgaaatatacgcaacgaactaacgc

SEQ ID NO: 28 - DNA, 44 bp, Artificial Sequence
  Description of Artificial Sequence Synthetic GSTP1 scorpion primer
ccggtcgcgaggttttcgaccggccgaaaaacgaaccgcgcgta

SEQ ID NO: 29 - DNA, 27 bp, Artificial Sequence
  Description of Artificial Sequence Synthetic GSTP1 scorpion antisense primer
gggcgggattatttttataaggttcgg

SEQ ID NO: 30 - DNA, 49 bp, Artificial Sequence
  Description of Artificial Sequence Synthetic RASSFIA scorpion primer
gccgcggtttcgttcggttcgcggccccgtacttcgctaactttaaacg

SEQ ID NO: 31 - DNA, 18 bp, Artificial Sequence
  Description of Artificial Sequence Synthetic RASSFIA scorpion antisense primer
gcgttgaagtcggggttc

SEQ ID NO: 32 - DNA, 47 bp, Artificial Sequence
  Description of Artificial Sequence Synthetic RARB2 scorpion primer
cggcgcccgacgatacccaaagcgccgaacgcgagcgattcgagtag

SEQ ID NO: 33 - DNA, 24 bp, Artificial Sequence
  Description of Artificial Sequence Synthetic RARB2 scorpion antisense primer
cttacaaaaaaccttccgaatacg

SEQ ID NO: 34 - DNA, 44 bp, Artificial Sequence
  Description of Artificial Sequence Synthetic APC scorpion primer
gccggcgggttttcgacgggccggccgaaccaaaacgctcccca

SEQ ID NO: 35 - DNA, 25 bp, Artificial Sequence
  Description of Artificial Sequence Synthetic APC scorpion antisense primer
gtcggttacgtgcgtttatatttag

SEQ ID NO: 36 - DNA, 44 bp, Artificial Sequence
  Description of Artificial Sequence Synthetic Actin scorpion primer
gcgcccaaccgcacaagggcgcgggtatattttcgaggggtacg

SEQ ID NO: 37 - DNA, 18 bp, Artificial Sequence
  Description of Artificial Sequence Synthetic ACTIN scorpion antisense primer
cgacccgcactccgcaat

SEQ ID NO: 38 - DNA, 52 bp, Artificial Sequence
  Description of Artificial Sequence Synthetic ACTIN scorpion primer
ccgcgcatcaccaccccacacgcgcggggagtatataggttggggaagtttg

SEQ ID NO: 39 - DNA, 27 bp, Artificial Sequence
  Description of Artificial Sequence Synthetic ACTIN scorpion antisense primer
aacacacaataacaaacacaaattcac

SEQ ID NO: 40 - DNA, 50 bp, Artificial Sequence
  Description of Artificial Sequence Synthetic ACTIN scorpion primer
cccggctaaacccaccatccagccggggggagggtagtttagttgtggtt

SEQ ID NO: 41 - DNA, 29 bp, Artificial Sequence
  Description of Artificial Sequence Synthetic ACTIN scorpion antisense primer
caaaacaaaaaaactaaatctacacaacc

SEQ ID NO: 42 - DNA, 47 bp, Artificial Sequence
  Description of Artificial Sequence Synthetic ACTIN scorpion primer
ccgcggaacattcaactcaaccgcggggaggaggaaggtaggttttt

SEQ ID NO: 43 - DNA, 29 bp, Artificial Sequence
  Description of Artificial Sequence Synthetic ACTIN scorpion antisense primer
acatacaacaatcaataacataaaaccac

SEQ ID NO: 44 - DNA, 47 bp, Artificial Sequence
  Description of Artificial Sequence Synthetic PTGS2/COX2 scorpion primer
cacgccgccgtatctaggcgtggtttgtttcgacgtgattttttcga

SEQ ID NO: 45 - DNA, 21 bp, Artificial Sequence
  Description of Artificial Sequence Synthetic PTGS2/COX2 antisense primer
gcaaaaaatcccctctcccgc

SEQ ID NO: 46 - DNA, 45 bp, Artificial Sequence
  Description of Artificial Sequence Synthetic PTGS2/COX2 scorpion primer
gccgcgcacaaatttccgcggcgaattggttttcggaagcgttcg

SEQ ID NO: 47 - DNA, 16 bp, Artificial Sequence
  Description of Artificial Sequence Synthetic PTGS2/COX2 antisense primer
cccgaattccaccgcc

SEQ ID NO: 48 - DNA, 46 bp, Artificial Sequence
  Description of Artificial Sequence Synthetic PTGS2/COX2 scorpion primer
ggcggaacgcacaaatttccgccgaattggttttcggaagcgttcg

SEQ ID NO: 49 - DNA, 16 bp, Artificial Sequence
  Description of Artificial Sequence Synthetic PTGS2/COX2 antisense primer
cccgaattccaccgcc

SEQ ID NO: 50 - DNA, 51 bp, Artificial Sequence
  Description of Artificial Sequence Synthetic PTGS2/COX2 scorpion primer
tgccgccgccgtatctaatggcggcagtttgtttcgacgtgattttttcga

SEQ ID NO: 51 - DNA, 21 bp, Artificial Sequence
  Description of Artificial Sequence Synthetic PTGS2/COX2 antisense primer
gcaaaaaatcccctctcccgc

SEQ ID NO: 52 - DNA, 39 bp, Artificial Sequence
  Description of Artificial Sequence Synthetic CDHI scorpion primer
cgccgaatacgatcggcggttcgttttagttcggttcga

SEQ ID NO: 53 - DNA, 18 bp, Artificial Sequence
  Description of Artificial Sequence Synthetic CDH1 scorpion antisense primer
accgaaaacgccgaacga

SEQ ID NO: 54 - DNA, 37 bp, Artificial Sequence
  Description of Artificial Sequence Synthetic 15LO1 scorpion primer
ggcggcgttcgggccgccccgtacgaaccacaatcgc

SEQ ID NO: 55 - DNA, 21 bp, Artificial Sequence
  Description of Artificial Sequence Synthetic 15LO1 scorpion antisense primer
ggggtttcgttttatgtcggt

SEQ ID NO: 56 - DNA, 2671 bp, Homo sapiens
  15LO1 gi|187190|gb|M23892.1|HUMLOX15A Human 15-lipoxygenase mRNA, complete cds
aagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaag

SEQ ID NO: 57 - DNA, 1193 bp, Homo sapiens
  CDH1 gi|509604|gb|L34545.1|HUMECADN Human E-cadherin gene, promoter region and 5′ end
tctagaaaaattttttaaaaaattaggccgctcgagcgagagtgcagtggctcacgcctgtaatccaacacttcaggaggctgaagagggtggatcacctgaggtcaggagttccagaccagcctggccaacatggtgaaaccccgtcttgtactaaaaatacaaaattagccggtgtggtggcacacgcctgtagtcccagctactcaataggctgagacaggagagtctcttgaacccggcaggcggaggttgcagtgagccgagatcgtgccactgcactccagcctgggcaagacagagcgagactccgtctcaaaaaatacaaacaaaacaaacaaacaaaaaattaggctgctagctcagtggctcatggctcacacctgaaatcctagcactttgggaggccaaggcaggaggatcgcttcagcccaggagttcgagaccaggctgggcaatacagggagacacagcgcccccactgcccctgtccgccccgacttgtctctctacaaaaaggcaaaagaaaaaaaaaattagcctggcgtggtggtgtgcacctgtactcccagctactagagaggctggggccagaggaccgcttgagcccaggagttcgaggctgcagtgagctgtgatcgcaccactgcactccagcttgggtgaaagagtgagccccatctccaaaacgaacaaacaaaaaatcccaaaaaacaaaagaactcagccaagtgtaaaagccctttctgatcccaggtcttagtgagccaccggcggggctgggattcgaacccagtggaatcagaaccgtgcaggtcccataacccacctagaccctagcaactccaggctagagggtcaccgcgtctatgcgaggccgggtgggcgggccgtcagctccgccctggggaggggtccgcgctgctgattggctgtggccggcaggtgaaccctcagccaatcagcggtacggggggcggtgctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcag

SEQ ID NO: 58 - DNA, 2635 bp, Homo sapiens
  Hin-1 region on chromosome 5 location 179,949,712-179,951,146 start of gene located in Contig AC122714.2.1.190024 chromosome NCBI355179949112 179951746-1; (Alternate name (SCGB3A1))
atctgtcatacttctgcaggtgacagacgagtgaggacatttagagaaactcaggaacaaaccaggcccatccactggcgtcgaggctagtttcgaagacagaaaaacgccccacttctgtttgcactgtggctgcctgtggcccggccggggaggcggccgggagtgaggcctgatcgtccctggcgcctccacctccccaggcgcagaaggcgcccacgaggacccccagtgcccgacgttgccacggtctgggatcagaggcagggaccagggagccaggaactgcgccgcccccgcccctgccctggcgcgagggaagctcccctcacccgggcccagccctgcaggggggcgcgtggggtcagaccgcaaagcgaaggtgcgggccggggtgggcctcgcggagacaaaggccgggcctgcctgctctcagagggccccagcgcctgccaagaggaagtcctcgaggcccgggcagggaagggggcacgggcttcccagggcccgccggccgcagcaggaagttggccagggcacggccgtgagcggagcgggcagggctttctcaggagcgcgggcgaggccggcgctggaggggcgaggaccgggtataagaagcctcgtggccttgcccgggcagccgcaggttccccgcgcgccccgagcccccgcgccatgaagctcgccgccctcctggggctctgcgtggccctgtcctgcagctccggtgagcgccccgggctcctggcgcgcacggtggggcctgaggcctcggcgcccgctgcgcccccgccgctgcctgcgccgatcgtggatcccaggtccttccagcctcgcccggcgcccagagggctccgccaccccggggccccgcgcctgcagcccgcggcctccccctctcagggctgtctccagcctcgtgccgggatggaggccgcccctggcctggggacacccgtctgccccccgtctggagaccggctcctcatcctgcaaggcgcagcgcgagtgtcccctcccttgggggcctgtcccggaccctgcacagagttcaccggtccttccgccaccctcaggccactccggtgaccctgcagcgtctcctggcggggccgcctccccagacccgcctgtgtcccgggggctcgcacctggcaggcctcggcgcaggagggaggggcggtcggggagccgggcagggcgcgacggttcctgggcgcctcccgcgggggcgcgtcctggacctgccttctggggaccccggcgcgcaggcggtgacccctccctgttcgcttgcagctgctgctttcttagtgggctcggccaagcctgtggcccagcctgtcgctgcgctggagtcggcggcggaggccggggccgggaccctggccaaccccctcggcaccctcaacccgctgaagctcctgctgagcagcctgggcatccccgtgaaccacctcatagagggctcccagaagtgtgtggctgagctgggtccccaggccgtgggggccgtgaaggccctgaaggccctgctggtaaagtgggcaccccgggtgcccttcctgcgcgggcatcccttcccggccagcctcaaaactgcaccagaggtgtcccggccctacgtcagggctgtttcccgtcctgcccctccaagccctgtccctgggagaagcccgggtctccgggtgggaactgggtggggcgcatccgagctgcaggggcggggaaggcggagatggccccgcgcgggtgcgcgtcccccggagacgccagcggaaccgccccgctcccgctccccacagagccggccgctgccgcgcccccgcctgagttcttggtggcagggctgggggcactcaagctcggcttctgctttccagggggccctgacagtgtttggctgagccgagactggagcatctacacctgaggacaagacgctgcccacccgcgagggctgaaaaccccgccgcggggaggaccgtccatccccttcccccggcccctctcaataaacgtggttaagagcagctcgagcctggtattttctcatccaaatgctgatccctgtgggggaccgagagtgccaagtcccagattttgtgaggggtgctgcgtgagagagggagagagaggccgcgagagtctgcaggtggcatagagccagcagtgcaaggtgacagagatggggccagtatcagaggtaaaccggatttggaaacaaaactggcaggtctttcttcctgttgtgctctgtaactgtttatgaagcataaaaattatctactccttgaagactggcgagaatgcaaacgcaccaaagccatttgttgctaaaatttgaaagtttttattttttccctctatacttgtagatttcttcctcggaatgggggaggaatgagggcttcaggaaatccgctgttgggcttgttttccctgcagtttctgctggttttgtccaaataacctagtgactttgattccttctactctagcggagagaccaccttttgtcaactgatctagtgttggtgggaaggtgcccagggaacccagacatcggtgtccctttcaggggaagcctcccagttgacttccttgctgggatgaataactctgcctcagg

SEQ ID NO: 59 - DNA, 4260 bp, Homo sapiens
  gi|341173|gb|M24485.1|HUMGSTP1G Homo sapiens (clone pHGST-pi) glutathione S-transferase pi (GSTP1) gene, complete cds
aacaagagatcaatatctagaataaatggagatctgcaaatcaacagaaagtaggcagcaaagccaaagaaaatagcctaaggcacagccactaaaaggaacgtgatcatgtcctttgcagggacatgggtggagctggaagccgttagcctcagcaaactcacacaggaacagaaaaccagcgagaccgcatggtctcacttataagtgggagctgaacaatgagaacacatggtcacatggcggcgatcaacacacactggtgcctgttgagcggggtgctggggagggagagtaccaggaagaatagctaagggatactgggcttaatacctgggtgatgggatgatctgtacagcaaaccatcatggcgcacacacctatgtaacaaacctgcacatcctgcacatgtaccccagaacttcaaataaaagttggacggccaggcgtggtggctcacgcctgtaatcccagcactttgggaagccgaggcgtgcagatcacctaaggtcaggagttcgagaccagcccggccaacatggtgaaaccccgtctctactaaaaatacaaaaatcagccagatgtggcacgcacctataattccacctactcgggaggctgaagcagaattgcttgaacccgagaggcggaggttgcagtgagccgccgagatcgcgccactgcactccagcctgggccacagcgtgagactacgtcataaaataaaataaaataacacaaaataaaataaaataaaataaaataaaataaaataataaaataaaataaaataaaataaaataaaataaaataaagcaatttcctttcctctaagcggcctccacccctctcccctgccctgtgaagcgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgctccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcaggctgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttacctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgcccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcaggcatgggcaagcctctgcccccggagcccttttgtttaaatcagctgccccgcagccctctggagtggaggaaactgagacccactgaggttacgtagtttgcccaaggtcaagcctgggtgcctgcaatccttgccctgtgccaggctgcctcccaggtgtcaggtgagctctgagcacctgctgtgtggcagtctctcatccttccacgcacatcctcttcccctcctcccaggctggggctcacagacagccccctggttggcccatccccagtgactgtgtgttgatcaggcgcccagtcacgcggcctgctcccctccacccaaccccagggctctatgggaaggaccagcaggaggcagccctggtggacatggtgaatgacggcgtggaggacctccgctgcaaatacatctccctcatctacaccaactatgtgagcatctgcaccagggttgggcactgggggctgaacaaagaaaggggcttcttgtgccctcaccccccttacccctcaggtggcttgggctgaccccttcttgggtcagggtgcaggggctgggtcagctctgggccaggggcccaggggcctgggacaagacacaacctgcacccttattgcctgggacatcaaccagccaagtaacgggtcatgggggcgagtgcaaggacagagacctccagcaactggtggtttctgatctcctggggtggcgagggcttcctggagtagccagaggtggaggaggatttgtcgccagtttctggatggaggtgctggcacttttagctgaggaaaatatgcagacacagagcacatttggggacctgggaccagttcagcagaggcagcgtgtgtgcgcgtgcgtgtgcgtgtgtgtgcgtgtgtgtgtgtacgcttgcatttgtgtcgggtgggtaaggagatagagatgggcgggcagtaggcccaggtcccgaaggccttgaacccactggtttggagtctcctaagggcaatgggggccattgagaagtctgaacagggctgtgtctgaatgtgaggtctagaaggatcctccagagaagccagctctaaagcttttgcaatcatctggtgagagaacccagcaaggatggacaggcagaatggaatagagatgagttggcagctgaagtggacaggatttggtactagcctggttgtggggagcaagcagaggagaatctgggactctggtgtctggcctggggcagacgggggtgtctcaggggctgggagggatgagagtaggatgatacatggtggtgtctggcaggaggcgggcaaggatgactatgtgaaggcactgcccgggcaactgaagccttttgagaccctgctgtcccagaaccagggaggcaagaccttcattgtgggagaccaggtgagcatctggccccatgctgttccttcctcgccaccctctgcttccagatggacacaggtgtgagccatttgtttagcaaagcagagcagacctaggggatgggcttaggccctctgcccccaattcctccagcctgctcccgctggctgagtccctagcccccctgccctgcagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagctactatgagcactgtgtttcctgggacggggcttaggggttctcagcctcgaggtcggtgggagggcagagcagaggactagaaaacagctcctccagcacagtcagtggcttcctggagccctcagcctggctgtgtttactgaacctcacaaactagaagaggaagaaaaaaaaagagagagagaaacaaagagaaat

SEQ ID NO: 60 - DNA, 2011 bp, Homo sapiens
  B-Actin gi|28337|emb|Y00474.1|HSACTBPR Human beta-actin gene 5′-flanking region, CpG Island 1656 to 1955
gagctctgtctcttggccagctgaatggaggcccagcggcaacacaggtcctgcctggggatcaggtctgctctgcaccccaccttgctgcctggagccgcccacctgacaacctctcatccctgctctgtagatccggtcccatccccactgcccaccccacccccccagcactccacccagttcaacgttccacgaacccccagaaccagccctcatcaacaggcagcaagaagggccccccgcccatcgccccacaacgccagccgggtgaactgtagcgttggcaggtcctgaggcagctgaaagatacaaggccagggacaggacagtcccatccccaggaggcagggagtatacaggctggggaagtttgcccttgcgtggggtggtgatggaggaggctcagcaagtcttctggactgtgaacctgtgtctgccactgtgtgctgggtggtggtcatctttcccaccaggctgtggcctctgcaaccttcaagggaggagcaggtcccattggctgagcacagccttgtacgtgaactgaacaagcagcctccttcctggccacaggttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccacttccttcccagggctgcggctgggtaggtttgtagccttcatcacgggccacctccagccactggaccgctggcccctgccctgtcctggggagtgtggtcctgcgactctaatggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtcggcgaagccggtgagtgagcggcgcggggccaatcgcgtgcgccgttccgaaagttgccttttatggctcgagcggccgcggcggcgccctataaaacccagcggcgcgacgcgccacc

SEQ ID NO: 61 - DNA, 1792 bp, Homo sapiens
  gi|5016088|ref|NM_001101.2| Homo sapiens actin, beta (ACTB), mRNA
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctt

SEQ ID NO: 62 - DNA, 2762 bp, Homo sapiens
  RARB2 gi|14916495|ref|NM_016152.2| Homo sapiens retinoic acid receptor, beta (RARB), transcript variant 2, mRNA
gtgacagaagtagtaggaagtgagctgttcagaggcaggagggtctattctttgccaaaggggggaccagaattccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatgcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagatgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccactggaccatgtaactctagtgtccttcctgattcatgcctgatattgggatttttttccagcccttcttgatgccaagggctaattatattacatcccaaagaaacaggcatagaatctgcctcctttgaccttgttcaatcactatgaagcagagtgaaagctgtggtagagtggttaacagatacaagtgtcagtttcttagttctcatttaagcactactggaatttttttttttgatatattagcaagtctgtgatgtactttcactggctctgtttgtacattgagattgtttgtttaacaatgctttctatgttcatatactgtttacctttttccatggactctcctggcaaagaataaaatatatttatttt

SEQ ID NO: 63 - DNA, 5487 bp, Homo sapiens
  TIMP3 gi|21536431|ref|NM_000362.3| Homo sapiens tissue inhibitor of metalloproteinase 3 (Sorsby fundus dystrophy, pseudoinflammatory)(TIMP3), mRNA
tctgtcgacttgccccagagctgatccttgtctttgtccacttctcagcgaggatggcacttcagggagcccttcccttactatcgcagagagagcaggccctccccagtcatgtccaacccagaactctgttttgttttcttcatagccctagcatcacagaaaatcaccctgtgcattcatggatgtccacgggggcaagggctttgtgttgcttaacccagcatcctgaaccgtgtttgttgaatgaatacagaaccccgtttgctctgggagagcacagaaaacagtcttctatcatatatcatagccagctgcaaacagcagatggcttcccatatcccagagagtaagaaccagagagagagagaaagagagagagtttgggtctttctcctctgtgcctgctctctccagagaaactggaggggtagcagttagcattcccccgctggttccaccaagcacagtcaaggtctctaggacatggccacccctcacctgtggaagcggtcctgctggggtgggtgggtgttagttggttctggtttgggtcagagacacccagtggcccaggtgggcgtggggccagggcgcagacgagaaggggcacgagggctccgctccgaggacccagcggcaagcaccggtcccgggcgcgccccagcccacccactcgcgtgcccacggcggcattattccctataaggatctgaacgatccgggggcggccccgccccgttaccccttgcccccggccccgccccctttttggagggccgatgaggtaatgcggctctgccattggtctgagggggcgggccccaacagcccgaggcggggtccccgggggcccagcgctatatcactcggccgcccaggcagcggcgcagagcgggcagcaggcaggcggcgggcgctcagacggcttctcctcctcctcttgctcctccagctcctgctccttcgccgggaggccgcccgccgagtcctgcgccagcgccgaggcagcctcgctgcgccccatcccgtcccgccgggcactcggagggcagcgcgccggaggccaaggttgccccgcacggcccggcgggcgagcgagctcgggctgcagcagccccgccggcggcgcgcacggcaactttggagaggcgagcagcagccccggcagcggcggcagcagcggcaatgaccccttggctcgggctcatcgtgctcctgggcagctggagcctgggggactggggcgccgaggcgtgcacatgctcgcccagccacccccaggacgccttctgcaactccgacatcgtgatccgggccaaggtggtggggaagaagctggtaaaggaggggcccttcggcacgctggtctacaccatcaagcagatgaagatgtaccgaggcttcaccaagatgccccatgtgcagtacatccacacggaagcttccgagagtctctgtggccttaagctggaggtcaacaagtaccagtacctgctgacaggtcgcgtctatgatggcaagatgtacacggggctgtgcaacttcgtggagaggtgggaccagctcaccctctcccagcgcaaggggctgaactatcggtatcacctgggttgtaactgcaagatcaagtcctgctactacctgccttgctttgtgacttccaagaacgagtgtctctggaccgacatgctctccaatttcggttaccctggctaccagtccaaacactacgcctgcatccggcagaagggcggctactgcagctggtaccgaggatgggcccccccggataaaagcatcatcaatgccacagacccctgagcgccagaccctgccccacctcacttccctcccttcccgctgagcttcccttggacactaactcttcccagatgatgacaatgaaattagtgcctgttttcttgcaaatttagcacttggaacatttaaagaaaggtctatgctgtcatatggggtttattgggaactatcctcctggccccaccctgccccttctttttggttttgacatcattcatttccacctgggaatttctggtgccatgccagaaagaatgaggaacctgtattcctcttcttcgtgataatataatctctatttttttaggaaaacaaaaatgaaaaactactccatttgaggattgtaattcccacccctcttgcttcttccccacctcaccatctcccagaccctcttccctttgcccttctcctccaatacataaaggacacagacaaggaacttgctgaaaggccaaccatttcaggatcagtcaaaggcagcaagcagatagactcaaggtgtgtgaaagatgttatacaccaggagctgccactgcatgtcccaaccagactgtgtctgtctgtgtctgcatgtaagagtgagggagggaaggaaggaactacaagagagtcggagatgatgcagcacacacacaattccccagcccagtgatgcttgtgttgaccagatgttcctgagtctggagcaagcacccaggccagaataacagagctttcttagttggtgaagacttaaacatctgcctgaggtcaggaggcaatttgcctgccttgtacaaaagctcaggtgaaagactgagatgaatgtctttcctctccctgcctcccaccagacttcctcctggaaaacgctttggtagatttggccaggagctttcttttatgtaaattggataaatacacacaccatacactatccacagatatagccaagtagatttgggtagaggatactatttccagaatagtgtttagctcacctagggggatatgtttgtatacacatttgcatatacccacatggggacataagctaatttttttacaggacacagaattctgttcaatgctgttaaatatgccaatagtttaatctcttctattttgttgtcgttgcttgtttgaagaaaatcatgacattccaagttgacatttttttttcattttaattaaaatttgaaattctgaacaccgtcagcaccctctcttccctatcatgggtcatctgacccctgtccgtctccttgtccctgcttcatgtttgggggcctttctttaactgccttcctggcttagctcagatggcagatgagagtgtagtcaagggcctgggcacaggagggagagctgcagagtgtcctgcctgccttggctggagggacacctctcctgggtgtggagacagcttggttccctttccctagctccctggtgggtgaatgccacctcctgagatcctcacctcttggaattaaaattgttggtcactggggaaagcctgagtttgcaaccagttgtagggtttctgttgtgtttttttttttttttttgaaataaaactataatataaattctcctattaaataaaattattttaagttttagtgtcaaaagtgagatgctgagagtaggtgataatgtatattttacagagtgggggttggcaggatggtgacattgaacatgattgctctctgtctcttttttcagcttatgggtatttatcttctattagtatttgtatcttcagttcattccactttaggaaacagagctgccaattgaaacagaagaagaaaaaaaaaaaaagcagcagacaacacactgtagagtcttgcacacacacaagtgcccaggcaaggtgcttggcagaaccgcagagtgggaagagagtaccggcatcgggtttccttgggatcaatttcattaccgtgtacctttcccattgtggtcatgccatttggcagggggagaatgggaggcttggccttctttgtgaggcagtgtgagcagaagctgatgccagcatgtcactggttttgaagggatgagcccagacttgatgttttgggattgtccttattttaacctcaaggtctcgcatggtggggcccctgaccaacctacacaagttccctcccacaagtggacatcagtgtcttctctgtgaggcatctggccattcgcactccctggtgtggtcagcctctctcacacaaggaggaacttgggtgaaggctgagtgtgaggcacctgaagtttccctgcggagtcgataaattagcagaaccacatccccatctgttaggccttggtgaggaggccctgggcaaagaagggtctttcgcaaagcgatgtcagagggcggttttgagctttctataagctatagctttgtttatttcacccgttcacttactgtataatttaaaatcatttatgtagctgagacacttctgtatttcaatcatatcatgaacattttattttgctaaatcttgtgtcatgtgtaggctgtaatatgtgtacattgtgtttaagagaaaaatgaaacccacatgccgccattttcctgaatcaaattctgcagtggaatggagaggaaaatacttctaggcaagcagctagactggtgaattgggggaaatagaaggaactagtaactgagactcctccagcctcctccctattggaatcccaatggctcctggagtaggaaaaaagtttaaactacattcatgttcttgttctgtgtcactcggccctgggtagtctaccatttacttcaccccaagtcctgctgcccatccagttgggaagccatgattttcctaagaatccagggccatgggagatacaattccaagttctcgcttcctcctttgggcatctcttctgcctcccaatcaaggaagctccatgctcaggctctcagctctcgggccagtgctctgctctgtccagggtaggtaatactgggagactcctgtcttttaccctcccctcgttccagacctgcctcatggtggcaacatggttcttgaacaattaaagaaacaaatgactttttggaatagccctgtctagggcaaactgtggcccccaggagacactacccttccatgccccagacctctgtcttgcatgtgacaattgacaatctggactaccccaagatggcacccaagtgtttggcttctggctacctaaggttaacatgtcactagagtatttttatgagagacaaacattataaaaatctgatggcaaaagcaaaacaaaatggaaagtaggggaggtggatgtgacaacaacttccaaattggctctttggaggcgagaggaaggggagaacttggagaatagtttttgctttgggggtagaggcttcttagattctcccagcatccgcctttccctttagccagtctgctgtcctgaaacccagaagtgatggagagaaaccaacaagagatctcgaaccctgtctagaaggaatgtatttgttgctaaatttcgtagcactgtttacagttttcctccatgttatttatgaattttatattccgtgaatgtatattgtcttgtaatgttgcataatgttcactttttatagtgtgtcctttattctaaacagtaaagtggttttatttctatcac

SEQ ID NO: 64 - DNA, 866 bp, Homo sapiens
  APC gi|551463|gb|U02509.1|HSU02509 Human adenomatous polyposis coli (APC) gene, promoter sequence
acttatatatctgacagttgatttgtcctcacctctaaattggaatttaagcatcacctggttcgatttaatgcaatgtagaatttgcattaaaatactacattaaagcctcagatttgtagtagctaacagcacttctatgtatgtgtcagggactgctctaaatacttcatatatattaactcctctattctgtacttctgttcccgttttatacagcaggaaattgaaacactgagaggttaagtaactaaagttacagagctagagtgacaggagtaaagcttcaactcaggcaacccagacgtccagagntctgatctccactactaagctgctagcatagcttttctggtaactatttttaattcaatataattcgaatgatctatctaacaagtcatcactctgacaactcagtgacttgtaatgtaaaattattcattgtaattcacttaatattattgtttctctgtgctgcaaaaatcatagcaatcgagatgtaatttattactctccctcccacctccggcatcttgtgctaatccttctgccctgcggacctcccccgactctttactatgcgtgtcaactgccatcaacttccttgcttgctggggactggggccgtgagggcatacccccgaggggtacggggctagggctaggcaggctgtgcggttgggcggggccctgtgccccactgcggagtgcgggtcgggaagcggagagagaagcagctgtgtaatccgctggatgcggaccagggcgctccccattcccgtcgggagcccgccgattggctgggtgtgggcgcacgtgaccgacatgtggctgtattggtgcagcccgccagggtgtcactg

SEQ ID NO: 65 - DNA, 10386 bp, Homo sapiens
  gi|21626462|ref|NM_000038.2| Homo sapiens adenomatosis polyposis coli (APC), mRNA
attgaggactcggaaatgaggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagctttacccagcctgtttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaattttttncttcagcttctatgcattaagagtaaaattcctcttactgtaataaaaacaattgaagaagactgttgccacttaaccattccatgcgttggcacttatctattcctgaaattcttttatgtgattagctcatcttgatttttaacatttttccacttaaacttttttttcttactccactggagctcagtaaaagtaaattcatgtaatagcaatgcaagcagcctagcacagactaagcattgagcataataggcccacataatttcctctttcttaatattatagaaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagagaaaacctttttaagcatggtggggcactcagataggagtgaatacacctacctggtggtcat

SEQ ID NO: 66 - DNA, 7273 bp, Homo sapiens
  PTGS2 gi|3282785|gb|AF044206.1| Homo sapiens cyclooxygenase (COX-2) gene, promoter and exon 1
ggtacccaggctggagtgcactggtgtgatcatagctcactaacctcgaactcctgggcttaggcaatcctcttgccttggcctcccaaagtgccaggattacaggcatgagccaccacagtggagctctcaattctgatactaataatttgtgtcttctctttttttccttagcctgactagagtaattaactttatgtcttttaaaagaaccacctttttggttttacccattttcttttttgattttctgtttttgatttgattgatatctactctaattttttattatttcttttcctctgcttactttgaatttaattacttttcttttttgtagtctcctaaaatagaagcttatattattgattttagatctttcttcttttctattacagcactcaatgctataaatttccctctaagcattgctttcactgcatcctacaatatttcaactctattgttatttagctcaaaagaggttcttaatttctattgggatttctctttgacccatgtgttattcagaagtgttccgtgtgatctccaaatatttgggagtttttcagctatctttctattaatcatttcttgtttaattctattgtggcctgagagcatatattgtatgatttatattcttgtaaatgtgttaaggtgtgtcttatggtgcagaatgcggtttatcttgctatatgttccttagagaataatgtatgttctgctgttattggataaagtagtctatagatgtcagttacatctcgttgattaatggtgctgttgagttcagctatgtcctaaatgattttctgtctgctgtatctgtctatttctgacacaaggctgttgaagtctccaaccataataatgaattaatctatttttctttgcagttttatcaattttgtcttatatatattgatgctccattgtttggcacatacacattaagaattgttatgtcttcttggagaatttacctttccataacatgtaacatttccctttattcctgataatttttcttgctcaaaagtttgccctgttggaaattaccagaactactctggctttatttgattagtgttagcatgctctctctttctctattcttacacttttaatgtatacttgactttgtatttaaagtggggttcttatagaaaacatatacttggtagggtgggaagtaaaataaaaagaaatacttgggtattggtttgatccactctaacaatctctatgttttaattgatgtatttagaccattgatacttatttttttatcctcatccctgtgattacccagagagctgcttaaattgattattgatatagacaaattaataattaatatctaccgtttgttactgttttctatttttcattgcccttactttctgctcctattttttgctcctttttctgttaatttaggttttgagttattttatatcattctattttctctcccttctcagcatatgaattatctttctttttgacttttttagtggctgccctgaaggttgcaatgtacatttacaaccagtcccaatctcctttcaaaaaacacaatactgtttcatggctagtgcaagtacctaataataagaagtcactcctaatttctttctctcattctttgtatctttactgttattcatttcacttgtacataagctgtaatctttcaatacattattgctattattatttcaaaacatgttatctattatatctatttaaaataagaaaaataggccaggtgcagtggcttactcatgtaatcccagcactttgggagaccgatggattgctagagctcaggaattcgagaccagcctgggcaacatagtgaaaccctgtctctactaaaaatacaaaaaaaaaaattgctgggcatggtggcatgggcctgtggtcacagctactcgggaggctgaggtgagaggattgcttgagcctgggaggcagaggttgcagtgaaccaaaatcaagctactgcactccagcctaagtgacagagtgagaccctgtctcaaaaaaaaaatgaaagaattatttttatttatcttcacttatttcttctctaatgctctttgtttctttagtatgtagatccaagtttctaacctgtatcatttttcttatctcaataacttcttttaacatttctcacaaagcagatctactggccacagaatgcctcaattttcatttgtctgagaaaaccttatttctccttcacttttgaaagataattttgtagggtacagaattctaggttgtaggttttttcccctcaaagtgaaatatttcattccactcttttcttctttgtatggtatctgagaagaagtcagatgtaattcttatcattattacttaaaagattgcttctgttcctttctctcttctccttcccttctttccttctctgtatattacaccttttatagttgccccatatttcttagatattatgttttggttttcttctgtgtttttttctttgattctcagttttagaagtctctatttatatatctgcaatcgcagggattctttcctctgccatgtccagtctactaataagcccttacagacattgttgacttctgttccagtgtttttgatctctagcatttctctgattatttcttggaattgccatctgtctacttacattaccaacctattcttgtgtgttgtcttatcatagtaattgcagttgttttaatttcataggtattgtaatttcaacatctctaccatatttgacattgattctgatgcttgctctgtcttatcaagctatgtttttgtcttttagtgtgacttctaattttttgttgaaagccaggcatgatgtactgagtgaaagaaactcaatacattgtaatgtgacgataagagttcaggggaagtgaagcattctatagtcctatagcaggtctcggccttttagtgagcctgtgcctatgaacggtgactttcaacaagtgcttttcattccactcttttcctgtccttaagtgggacaagatcactgggggggggctagaattgggtatttcccttctccaatgtagaagctaaagagagggctggagttgggtatttttcttcccctgtatggaaagctagaggcagttaaatttggatattttccttcttctaattcagttaggctgcgacaaaaatcccgacagtttaggctctaatattataaaataatttctcttgagtataggccttattaagaacactatactctgatggagctgagggggagttttctctgatattcactgcgagaacctcgtagagctccaggaagcaaaactcacaaaagtgtgggagtcttccagaatttttcctttgcagacttatctgcactgaacctccagaaattcatcaattacagttcaggttttcctacccaggtactggttttcatggaggtttctgcctgtgcatttctgctccagtaagttgttcttcttgtatggtctgtctttcaaattttttaagtagggttatgacctgtcgcctcacttctctgacagttctgagagtgttgatttttcagtttgcttagatttttacttgtttttaggatgaagtgacaatttccaagctcctccctgacatgccagatcagaaactgaaagtcctaagcctcatattctgtgcgtgggtatgttcacatcctgcctgctccagtgcccccacctcacactctctttcccttccttgtccccttgtgagatttctaggtccaatacaaagactgtgttcaactcattcaactacttggctcatctgagtattataatgaacaatcacaaaaaaaaatgaagtaaaagaaaaatccatcaaagaattgagatatttgagaaaaagaaaggagatcagtgttttataaaacttagaaatagattttttaagtgtttcttcattgacttatgtgaaaggacttttcttaatttaacaaattatgtgctttcgtttatagcctcaaaacttcttgtgtagctaagaatgggtaaataatcaggctttactaaaggactaacgtaaagatcttctgtaagtaacatttctgctactcaaggaagagataaacttcatggcataaccttgccaaagtatactaagaataaccctgacacaaagctcttttttcagccaacatgccatgaaagaaagaagacaaggggtgatctccactctctaagtgaaccactaaacccaccaaagaagaaacgagggaaatagaaagaggacccttgcctgagataatggatctgtatgtatgagtagtagaaccctgctcaaagtacaaggaagggaaaaaaaagttagtttatttggaattttggacattaagagtctttattgttcattttcttttaactcacatgaatggcttatcacttcaattaataaatatttcatttcttttcaatctatattcatgaaacaaatctgaaatgaacagtgcaacatgtgaatgtttagaacattataaaattaaacacaaaatctgtctggcaatcttcctagcatcttaggaaaaaagttgacaaaatttcaagcagcagaagggggcagtaaaactcaacagaaagctctggaagatttttaagattcttccttattttcttttcatgtagagtatttcccaacaaatttcagacgctaatagaaattttgtacaacagatccatatatttgcctaaaatagacacagaaacattgatatatgcaaacatgagagctataagttttacatgatcaaaaccttttttttatggtacacaatagtcacagtacttttccatataaaacaggtttagtggtcttaatttagtttggcacatttaatacactcccatgaccagcatcccaaatgtacctatccgttttattttattgtctcagaattgtcagttatttaataaattatgtaacttttttccttatgctcagatttgcacttctttctaaaactctgcccatccttaaagtcccagattctccttgaacttttttttttgactttccaagtacatggaactcttcactctatcctgctatataagtgacagaatttccactatgggatagatggagttcaattcctttgagtttaaaataatctaaatataattattccttatgccctgtttttccctcacttttgtatccaaatctcttttcagacaacagaacaattaatgtctgataaggaagacaatgatgatgatcacttcaaaataagcttgaattcaggattgtaatgtaaaattttagtactctctcacagtatggattctaacatggcttctaacccaaactaacattagtagctctaactataaacttcaaatttcagtagatgcaacctactcctttaaaatgaaacagaagattgaaattattaaattatcaaaaagaaaatgatccacgctcttagttgaaatttcatgtaagattccatgcaataaataggagtgccataaatggaatgatgaaatatgactagaggaggagaaaggcttcctagatgagatggaattttagtcatccgtgtctcatgaagaatcagatgtgtacactaagcaaaacagttaaaaaaaaaacctccaagtgagtctcttatttatttttttcttataagacttctacaaattgaggtacctggtgtagttttatttcaggttttatgctgtcattttcctgtaatgctaaggacttaggacataactgaattttctattttccacttcttttctggtgtgtgtgtatatatatatgtatatatacacacacacatatacatatatatatttttagtatctcaccctcacatgctcctccctgagcactacccatgatagatgttaaacaaaagcaaagatgaaattccaactgttaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtcctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctaccccccgcgccgcgccctgcccgaagctt

SEQ ID NO: 67 - DNA, 4465 bp, Homo sapiens
  gi|4506264|ref|NM_000963.1| Homo sapiens prostaglandin-endoperoxide synthase 2 (prostaglandin G/H synthase and cyclooxygenase) (PTGS2), mRNA
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 68 - DNA, 1336 bp, Homo sapiens
  14-3-3-Sigma gi|45238846|ref|NM_006142.3| Homo sapiens stratifin (SFN), mRNA
gagagacacagagtccggcattggtcccaggcagcagttagcccgccgcccgcctgtgtgtccccagagccatggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctgagtgttgcccgccaccgccccgccctgccccctccagtcccccaccctgccgagaggactagtatggggtgggaggccccacccttctcccctaggcgctgttcttgctccaaagggctccgtggagagggactggcagagctgaggccacctggggctggggatcccactcttcttgcagctgttgagcgcacctaaccactggtcatgcccccacccctgctctccgcacccgcttcctcccgaccccaggaccaggctacttctcccctcctcttgcctccctcctgcccctgctgcctctgatcgtaggaattgaggagtgtcccgccttgtggctgagaactggacagtggcaggggctggagatgggtgtgtgtgtgtgtgtgtgtgtgtgtgtgtgtgtgcgcgcgcgccagtgcaagaccgagattgagggaaagcatgtctgctgggtgtgaccatgtttcctctcaataaagttcccctgtgacactcaaaaaaaaaaaaaaaaaaaaa

SEQ ID NO: 69 - DNA, 1968 bp, Homo sapiens
  RASSF1A gi|25777678|ref|NM_007182.4| Homo sapiens Ras association (RalGDS/AF-6) domain family 1 (RASSF1), transcript variant A,mRNA
tctcctcagctccttcccgccgcccagtctggatcctgggggaggcgctgaagtcggggcccgccctgtggccccgcccggcccgcgcttgctagcgcccaaagccagcgaagcacgggcccaaccgggccatgtcgggggagcctgagctcattgagctgcgggagctggcacccgctgggcgcgctgggaagggccgcacccggctggagcgtgccaacgcgctgcgcatcgcgcggggcaccgcgtgcaaccccacacggcagctggtccctggccgtggccaccgcttccagcccgcggggcccgccacgcacacgtggtgcgacctctgtggcgacttcatctggggcgtcgtgcgcaaaggcctgcagtgcgcgcattgcaagttcacctgccactaccgctgccgcgcgctcgtctgcctggactgttgcgggccccgggacctgggctgggaacccgcggtggagcgggacacgaacgtggacgagcctgtggagtgggagacacctgacctttctcaagctgagattgagcagaagatcaaggagtacaatgcccagatcaacagcaacctcttcatgagcttgaacaaggacggttcttacacaggcttcatcaaggttcagctgaagctggtgcgccctgtctctgtgccctccagcaagaagccaccctccttgcaggatgcccggcggggcccaggacggggcacaagtgtcaggcgccgcacttccttttacctgcccaaggatgctgtcaagcacctgcatgtgctgtcacgcacaagggcacgtgaagtcattgaggccctgctgcgaaagttcttggtggtggatgacccccgcaagtttgcactctttgagcgcgctgagcgtcacggccaagtgtacttgcggaagctgttggatgatgagcagcccctgcggctgcggctcctggcagggcccagtgacaaggccctgagctttgtcctgaaggaaaatgactctggggaggtgaactgggacgccttcagcatgcctgaactacataacttcctacgtatcctgcagcgggaggaggaggagcacctccgccagatcctgcagaagtactcctattgccgccagaagatccaagaggccctgcacgcctgcccccttgggtgacctcttgtacccccaggtggaaggcagacagcaggcagcgccaagtgcgtgccgtgtgagtgtgacagggccagtggggcctgtggaatgagtgtgcatggaggccctcctgtgctgggggaatgagcccagagaacagcgaagtagcttgctccctgtgtccacctgtgggtgtagccaggtatggctctgcacccctctgccctcattactgggccttagtgggccagggctgccctgagaagctgctccaggcctgcagcaggagtggtgcagacagaagtctcctcaatttttgtctcagaagtgaaaatcttggagaccctgcaaacagaacagggtcatgtttgcaggggtgacggccctcatctatgaggaaaggttttggatcttgaatgtggtctcaggatatccttatcagagctaagggtgggtgctcagaataaggcaggcattgaggaagagtcttggtttctctctacagtgccaactcctcacacaccctgaggtcagggagtgctggctcacagtacagcatgtgccttaatgcttcatatgaggaggatgtccctgggccagggtctgtgtgaatgtgggcactggcccaggttcataccttatttgctaatcaaagccagggtctctccctcaggtgttttttatgaagtgcgtgaatgtatgtaatgtgtggtggcctcagctgaatgcctcctgtggggaaaggggttggggtgacagtcatcatcagggcctggggcctgagagaattggctcaataaagatttcaagatcctcaaaaaaaaaaaaaaaaaaa